SEQUENCE LISTING (2 sequences)

SEQ ID NO: 1 - DNA, 50 bp, Artificial Sequence
  16S_V3_F
  misc_feature (42)..(42): any nucleotide
tcgtcggcagcgtcagatgtgtataagagacagcctacgggnggcwgcag

SEQ ID NO: 2 - DNA, 55 bp, Artificial Sequence
  16S_V4_R
gtctcgtgggctcggagatgtgtataagagacaggactachvgggtatctaatcc

(19) United States
(12) Patent Application Publication
(10) Pub. No.: US 2020/0157632 A1
(43) Pub. Date: May 21, 2020

(54) METHOD OF DIAGNOSING GASTRIC CANCER THROUGH BACTERIAL METAGENOME ANALYSIS

(71) Applicant: MD HEALTHCARE INC., Seoul (KR)

(72) Inventor: Yoon-Keun Kim, Gyeonggi-do (KR)

(21) Appl. No.: 16/469,212

(22) PCT Filed: Dec. 15, 2017
(86) PCT No.: PCT/KR2017/014860

§ 371 Date: Jun. 13, 2019

(30) Foreign Application Priority Data
Dec. 16, 2016 (KR) 10-2016-0172584
Jun. 9, 2017 (KR) 10-2017-0072432

(51) Int. Cl. C12Q 1/6886; C12Q 1/689

(52) U.S. Cl. CPC C12Q 1/6886; C12Q 2600/158; C12Q 2531/113; C12Q 1/689

(57) ABSTRACT

The present invention relates to a method of providing information for gastric cancer diagnosis through bacterial metagenomic analysis, more particularly, a method of predicting a risk for gastric cancer or diagnosing gastric cancer by analyzing an increase or decrease in content of extracellular vesicles derived from specific bacteria through bacterial metagenomic analysis of genomes present in extracellular vesicles isolated from a subject-derived sample. Extracellular vesicles secreted from bacteria existing in the environment are absorbed into the human body, and thus may directly affect the occurrence of cancer, and gastric cancer has a very high incidence rate and a very high mortality rate in Korea, and thus prevention and early diagnosis thereof through prediction of the onset thereof is very important. Thus, according to the present invention, gastric cancer may be predicted through bacterial metagenomic analysis of extracellular vesicles present in a human body-derived sample, which leads to early diagnosis and prediction of a high risk group for gastric cancer, and, accordingly, the onset of gastric cancer may be delayed or gastric cancer may be prevented through appropriate management, and, even after gastric cancer occurs, early diagnosis for gastric cancer may be implemented, thereby lowering the onset of gastric cancer and increasing therapeutic effects. In addition, patients diagnosed with gastric cancer are able to avoid exposure to causative factors, whereby the progression of gastric cancer may be ameliorated, or recurrence of gastric cancer may be prevented.

### TECHNICAL FIELD

The present invention relates to a method of providing information for gastric cancer diagnosis through bacterial metagenomic analysis, and more particularly, to a method of predicting a risk for gastric cancer or diagnosing gastric cancer by analyzing an increase or decrease in content of extracellular vesicles derived from specific bacteria by bacterial metagenomic analysis of a genome present in extracellular vesicles isolated from a subject-derived sample.

### BACKGROUND ART

Globally, gastric cancer has a high incidence rate in the East Asia region including South Korea, China, Japan, and the like, while having a relatively low incidence rate in western countries including the USA, Europe, and the like. In South Korea, gastric cancer has the highest incidence rate among men and women, has the second highest mortality rate after lung cancer, and has the highest incidence rate in people in their 60s. Although non-examples of gastric cancer include gastric adenocarcinoma occurring in the gastric mucosal epithelium, malignant lymphoma occurring in the submucosal layer, muscle sarcomas, and interstitial tumors, gastric adenocarcinoma accounts for 95% of all gastric cancers. The stomach is an organ receiving food from the mouth and brought into contact therewith for a long period of time, and thus factors contained in foods are highly expected to be causative factors of gastric cancer, and carcinogens contained in foods are known as the most critical factors of gastric cancer through animal testing. It has long been demonstrated that chronic inflammation caused by biological factors such as viruses, bacteria, and the like causes cancer. It has recently been reported that colorectal cancer is caused by Th17 immune responses by toxins derived from bacteria living in the intestines and inflammatory responses thereby (Nat Commun. 2015 Apr. 24; 6:6956), and gastric cancer is caused by Helicobacter pylori known to coexist in the stomach.

Gastric cancer can be detected early through a regular checkup such as an endoscopy or the like, and early gastric cancer is expected to be completely cured in about 90% of the cases through appropriate treatment. However, there are still many cases in which gastric cancer is found after progression thereof and gastric cancer is also classified as cancer with a high mortality rate. Thus, it is important to differentiate coping methods for early diagnosis and treatment by predicting the onset of gastric cancer, and research thereon and technology development thereof are required.

Meanwhile, it is known that the number of microorganisms symbiotically living in the human body is 100 trillion which is 10 times the number of human cells, and the number of genes of microorganisms exceeds 100 times the number of human genes. A microbiota or microbiome is a microbial community that includes bacteria, archaea, and eukaryotes present in a given habitat. The intestinal microbiota is known to play a vital role in human's physiological phenomena and significantly affect human health and diseases through interactions with human cells. Bacteria coexisting in human bodies secrete nanometer-sized vesicles to exchange information about genes, proteins, and the like with other cells. The mucous membranes form a physical barrier membrane that does not allow particles with the size of 200 nm or more to pass therethrough, and thus bacteria symbiotically living in the mucous membranes are unable to pass therethrough, but bacteria-derived extracellular vesicles have a size of approximately 100 nm or less and thus relatively freely pass through the mucous membranes and are absorbed into the human body.

Metagenomics, also called environmental genomics, is analytics for metagenomic data obtained from samples collected from the environment. Recently, the bacterial composition of human microbiota has been listed using a method based on 16s ribosomal RNA (16s rRNA) base sequences, and microorganisms are identified by analyzing base sequences of bacteria through a next generation sequencing (NGS) platform. However, as for the occurrence of gastric cancer, there is no report about a method of identifying, from a human derived-fluid such as blood, stool, urine, or the like, a causative factor of gastric cancer by analysis of metagenomes present in bacteria-derived vesicles and of predicting gastric cancer.

### DISCLOSURE

### Technical Problem

To diagnose gastric cancer, the inventors of the present invention isolated extracellular vesicles from a subject-derived sample, such as blood, urine, and stool, extracted DNA therefrom, and performed bacterial metagenomic analysis on the extracted DNA, and, as a result, identified bacteria-derived extracellular vesicles having exhibited a significant increase or decrease in a gastric cancer patient-derived sample and thus being capable of acting as a causative factor or diagnosis biomarker for gastric cancer, thus completing the present invention.

Therefore, the present invention aims to provide a method of providing information for gastric cancer diagnosis by bacterial metagenomic analysis for DNA present in bacteria-derived extracellular vesicles.

However, the technical goals of the present invention are not limited to the aforementioned goals, and other unmentioned technical goals will be clearly understood by those of ordinary skill in the art from the following description.

### Technical Solution

According to an aspect of the present invention, there is provided a method of providing information for gastric cancer diagnosis, comprising the following processes:

(a) extracting DNA from extracellular vesicles isolated from a subject sample;

(b) performing polymerase chain reaction (PCR) on the extracted DNA using a pair of primers having SEQ ID NO:1 and SEQ ID NO: 2; and

(c) comparing an increase or decrease in content of bacteria-derived extracellular vesicles of the subject sample with that of a normal individual-derived sample through sequencing of a product of the PCR.

The present invention also provides a method of diagnosing gastric cancer, comprising the following processes:

(a) extracting DNA from extracellular vesicles isolated from a subject sample;

(b) performing PCR on the extracted DNA using a pair of primers having SEQ ID NOS: 1 and 2; and

(c) comparing an increase or decrease in content of bacteria-derived extracellular vesicles of the subject sample with that of a normal individual-derived sample through sequencing of a product of the PCR.

The present invention also provides a method of predicting a risk for gastric cancer, comprising the following processes:

(a) extracting DNA from extracellular vesicles isolated from a subject sample;

(b) performing PCR on the extracted DNA using a pair of primers having SEQ ID NOS: 1 and 2; and

(c) comparing an increase or decrease in content of bacteria-derived extracellular vesicles of the subject sample with that of a normal individual-derived sample through sequencing of a product of the PCR.

In one embodiment of the present invention, in process (c), the comparing may comprise comparing an increase or decrease in content of extracellular vesicles derived from one or more bacteria selected from the group consisting of the phylum Verrucomicrobia and the phylum Cyanobacteria that are isolated from a subject urine sample; and the phylum Tenericutes and the phylum Cyanobacteria that are isolated from a subject stool sample.

In one embodiment of the present invention, in process (c), the comparing may comprise comparing an increase or decrease in content of extracellular vesicles derived from one or more bacteria selected from the group consisting of the class Verrucomicrobiae and the class Chloroplast that are isolated from a subject urine sample; and the class Mollicutes that is isolated from a subject stool sample.

In one embodiment of the present invention, in process (c), the comparing may comprise comparing an increase or decrease in content of extracellular vesicles derived from one or more bacteria selected from the group consisting of the order Cardiobacteriales that is isolated from a subject blood sample; the order RF39, the order Stramenopiles, the order Verrucomicrobiales, the order Sphingomonadales, the order Bifidobacteriales, the order Streptophyta, and the order Aeromonadales that are isolated from a subject urine sample; and the order RF39, the order Neisseriales, and the order Enterobacteriales that are isolated from a subject stool sample.

In one embodiment of the present invention, in process (c), the comparing may comprise comparing an increase or decrease in content of extracellular vesicles derived from one or more bacteria selected from the group consisting of the family Methylocystaceae, the family Exiguobacteraceae, the family Peptostreptococcaceae, the family Brevibacteriaceae, the family Mogibacteriaceae, the family Acetobacteraceae, the family Rikenellaceae, and the family Leuconostocaceae that are isolated from a subject blood sample; the family Exiguobacteraceae, the family Porphyromonadaceae, the family Prevotellaceae, the family Verrucomicrobiaceae, the family Sphingomonadaceae, the family Bifidobacteriaceae, the family Methylobacteriaceae, the family Planococcaceae, and the family Comamonadaceae that are isolated from a subject urine sample; and the family Peptostreptococcaceae, the family Neisseriaceae, the family Enterobacteriaceae, the family Staphylococcaceae, the family Oxalobacteraceae, the family Moraxellaceae, and the family Planococcaceae that are isolated from a subject stool sample.

In one embodiment of the present invention, in process (c), the comparing may comprise comparing an increase or decrease in content of extracellular vesicles derived from one or more bacteria selected from the group consisting of the genus Cupriavidus, the genus Proteus, the genus Atopobium, the genus Micrococcus, the genus Odoribacter, the genus Faecalibacterium, the genus Veillonella, the genus Citrobacter, the genus Delftia, the genus Weissella, and the genus Leuconostocthat are isolated from a subject blood sample; the genus Morganella, the genus Rhizobium, the genus Exiguobacterium, the genus Proteus, the genus Parabacteroides, the genus Adlercreutzia, the genus Prevotella, the genus Acinetobacter, the genus Akkermansia, the genus Oscillospira, the genus Bifidobacterium, the genus Faecalibacterium, the genus Ruminococcus, the genus Coprococcus, the genus Pediococcus, and the genus Citrobacter that are isolated from a subject urine sample; and the genus Cupriavidus, the genus Proteus, the genus Methylobacterium, the genus Faecalibacterium, the genus Neisseria, the genus Staphylococcus, and the genus Acinetobacter that are isolated from a subject stool sample.

In one embodiment of the present invention, the subject sample may be blood, urine, or stool.

In one embodiment of the present invention, the blood may be whole blood, serum, plasma, or blood mononuclear cells.

### Advantageous Effects

Extracellular vesicles secreted from bacteria existing in the environment are absorbed into the human body, and thus may directly affect the occurrence of cancer, and gastric cancer has a very high incidence rate and a very high mortality rate in Korea, and thus prevention and early diagnosis thereof through prediction of the onset thereof is very important. Thus, according to the present invention, a risk for gastric cancer can be predicted through bacterial metagenomic analysis of genomes in extracellular vesicles present in a human body-derived sample, and thus the onset of gastric cancer can be delayed or gastric cancer can be prevented through appropriate management by early diagnosis and prediction of a risk group for gastric cancer , and, even after gastric cancer occurs, early diagnosis for gastric cancer can be implemented, thereby lowering a disease rate and increasing therapeutic effects. In addition, causative factors can be predicted by performing metagenomic analysis on patients diagnosed with gastric cancer, and thus the patients are able to avoid exposure to the causative factors, whereby the progression of gastric cancer is ameliorated, or recurrence of gastric cancer can be prevented.

### DESCRIPTION OF DRAWINGS

FIGS. 1A and 1B are views for evaluating the distribution pattern of extracellular vesicles derived from bacteria in vivo. FIG. 1A illustrates images showing the distribution pattern of intestinal bacteria and extracellular vehicles (EVs) derived from bacteria per time (0 h, 5 min, 3 h, 6 h, and 12 h) after being orally administered to mice. FIG. 1B illustrates images showing the distribution pattern of gut bacteria and EVs derived from bacteria after being orally administered to mice and, after 12 hours, blood and various organs (heart, lung, liver, kidney, spleen, adipose tissue, and muscle) of the mice were extracted.

FIG. 2 illustrates distribution results of bacteria-derived EVs exhibiting significant diagnostic performance at an order level, after metagenomic analysis of bacteria-derived EVs isolated from gastric cancer patient-derived blood and normal individual-derived blood.

FIG. 3 illustrates distribution results of bacteria-derived EVs exhibiting significant diagnostic performance at a family level, after metagenomic analysis of bacteria-derived EVs isolated from gastric cancer patient-derived blood and normal individual-derived blood.

FIG. 4 illustrates distribution results of bacteria-derived EVs exhibiting significant diagnostic performance at a genus level, after metagenomic analysis of bacteria-derived EVs isolated from gastric cancer patient-derived blood and normal individual-derived blood.

FIG. 5 illustrates distribution results of bacteria-derived EVs exhibiting significant diagnostic performance at a phylum level, after metagenomic analysis of bacteria-derived EVs isolated from gastric cancer patient-derived urine and normal individual-derived urine.

FIG. 6 illustrates distribution results of bacteria-derived EVs exhibiting significant diagnostic performance at a class level, after metagenomic analysis of bacteria-derived EVs isolated from gastric cancer patient-derived urine and normal individual-derived urine.

FIG. 7 illustrates distribution results of bacteria-derived EVs exhibiting significant diagnostic performance at an order level, after metagenomic analysis of bacteria-derived EVs isolated from gastric cancer patient-derived urine and normal individual-derived urine.

FIG. 8 illustrates distribution results of bacteria-derived EVs exhibiting significant diagnostic performance at a family level, after metagenomic analysis of bacteria-derived EVs isolated from gastric cancer patient-derived urine and normal individual-derived urine.

FIG. 9 illustrates distribution results of bacteria-derived EVs exhibiting significant diagnostic performance at a genus level, after metagenomic analysis of bacteria-derived EVs isolated from gastric cancer patient-derived urine and normal individual-derived urine.

FIG. 10 illustrates distribution results of bacteria-derived EVs exhibiting significant diagnostic performance at a phylum level, after metagenomic analysis of bacteria-derived EVs isolated from gastric cancer patient-derived stool and normal individual-derived stool.

FIG. 11 illustrates distribution results of bacteria-derived EVs exhibiting significant diagnostic performance at a class level, after metagenomic analysis of bacteria-derived EVs isolated from gastric cancer patient-derived stool and normal individual-derived stool.

FIG. 12 illustrates distribution results of bacteria-derived EVs exhibiting significant diagnostic performance at an order level, after metagenomic analysis of bacteria-derived EVs isolated from gastric cancer patient-derived stool and normal individual-derived stool.

FIG. 13 illustrates distribution results of bacteria-derived EVs exhibiting significant diagnostic performance at a family level, after metagenomic analysis of bacteria-derived EVs isolated from gastric cancer patient-derived stool and normal individual-derived stool.

FIG. 14 illustrates distribution results of bacteria-derived EVs exhibiting significant diagnostic performance at a genus level, after metagenomic analysis of bacteria-derived EVs isolated from gastric cancer patient-derived stool and normal individual-derived stool.

### BEST MODE

The present invention relates to a method of diagnosing gastric cancer through bacterial metagenomic analysis. The inventors of the present invention extracted genes from extracellular vesicles present in subject-derived samples such as blood, urine, stool, and the like, performed bacterial metagenomic analysis thereon, and identified bacteria-derived extracellular vesicles capable of acting as a causative factor of gastric cancer.

Thus, the present invention provides a method of providing information on gastric cancer diagnosis, the method comprising:

(a) extracting DNA from extracellular vesicles isolated from a subject sample;

(b) performing polymerase chain reaction (PCR) on the extracted DNA using a pair of primers having SEQ ID NOS: 1 and 2; and

(c) comparing an increase or decrease in content of bacteria-derived extracellular vesicles of the subject sample with that of a normal individual-derived sample through sequencing of a product of the PCR.

The term “gastric cancer diagnosis” as used herein refers to determining whether a patient has a risk for gastric cancer, whether the risk for gastric cancer is relatively high, or whether gastric cancer has already occurred. The method of the present invention may be used to delay the onset of gastric cancer through special and appropriate care for a specific patient, which is a patient having a high risk for gastric cancer or prevent the onset of gastric cancer. In addition, the method may be clinically used to determine treatment by selecting the most appropriate treatment method through early diagnosis of gastric cancer.

The term “metagenome” as used herein refers to the total of genomes including all viruses, bacteria, fungi, and the like in isolated regions such as soil, the intestines of animals, and the like, and is mainly used as a concept of genomes that explains identification of many microorganisms at once using a sequencer to analyze non-cultured microorganisms. In particular, a metagenome does not refer to a genome of one species, but refers to a mixture of genomes, including genomes of all species of an environmental unit. This term originates from the view that, when defining one species in a process in which biology is advanced into omics, various species as well as existing one species functionally interact with each other to form a complete species. Technically, it is the subject of techniques that analyzes all DNAs and RNAs regardless of species using rapid sequencing to identify all species in one environment and verify interactions and metabolism. In the present invention, bacterial metagenomic analysis is performed using extracellular vesicles isolated from, for example, blood and urine.

The term “bacteria-derived extracellular vesicles” as used herein collectively refers to membrane-formed nanoscale substances secreted by bacteria and archaea.

In the present invention, the subject sample may be blood, urine, or stool, and the blood may be whole blood, serum, plasma, or blood mononuclear cells, but is not limited to the above examples.

In an embodiment of the present invention, metagenomic analysis is performed on the bacteria-derived extracellular vesicles, and the bacteria-derived extracellular vesicles are actually identified as a biomarker for risk factors of gastric cancer and gastric cancer diagnosis by analysis at phylum, class, order, family, and genus levels.

More particularly, in one embodiment of the present invention, as a result of performing bacterial metagenomic analysis on extracellular vesicles present in subject-derived blood samples at an order level, the content of extracellular vesicles derived from bacteria belonging to the order Cardiobacteriales was significantly different between gastric cancer patients and normal individuals (see Example 4).

More particularly, in one embodiment of the present invention, as a result of performing bacterial metagenomic analysis on extracellular vesicles present in subject-derived blood samples at a family level, the content of extracellular vesicles derived from bacteria belonging to the family Methylocystaceae, the family Exiguobacteraceae, the family Peptostreptococcaceae, the family Brevibacteriaceae, the family Mogibacteriaceae, the family Acetobacteraceae, the family Rikenellaceae, and the family Leuconostocaceae was significantly different between gastric cancer patients and normal individuals (see Example 4).

More particularly, in one embodiment of the present invention, as a result of performing bacterial metagenomic analysis on extracellular vesicles present in subject-derived blood samples at a genus level, the content of extracellular vesicles derived from bacteria belonging to the genus Cupriavidus, the genus Proteus, the genus Atopobium, the genus Micrococcus, the genus Odoribacter, the genus Faecalibacterium, the genus Veillonella, the genus Citrobacter, the genus Delftia, the genus Weissella, and the genus Leuconostoc was significantly different between gastric cancer patients and normal individuals (see Example 4).

More particularly, in one embodiment of the present invention, as a result of performing bacterial metagenomic analysis on extracellular vesicles present in subject-derived urine samples at a phylum level, the content of extracellular vesicles derived from bacteria belonging to the phylum Verrucomicrobia and the phylum Cyanobacteria was significantly different between gastric cancer patients and normal individuals (see Example 5).

More particularly, in one embodiment of the present invention, as a result of performing bacterial metagenomic analysis on extracellular vesicles present in subject-derived urine samples at a class level, the content of extracellular vesicles derived from bacteria belonging to the class Verrucomicrobiae and the class Chloroplast was significantly different between gastric cancer patients and normal individuals (see Example 5).

More particularly, in one embodiment of the present invention, as a result of performing bacterial metagenomic analysis on extracellular vesicles present in subject-derived urine samples at an order level, the content of extracellular vesicles derived from bacteria belonging to the order RF39, the order Stramenopiles, the order Verrucomicrobiales, the order Sphingomonadales, the order Bifidobacteriales, the order Streptophyta, and the order Aeromonadales was significantly different between gastric cancer patients and normal individuals (see Example 5).

More particularly, in one embodiment of the present invention, as a result of performing bacterial metagenomic analysis on extracellular vesicles present in subject-derived urine samples at a family level, the content of extracellular vesicles derived from bacteria belonging to the family Exiguobacteraceae, the family Porphyromonadaceae, the family Prevotellaceae, the family Verrucomicrobiaceae, the family Sphingomonadaceae, the family Bifidobacteriaceae, the family Methylobacteriaceae, the family Planococcaceae, and the family Comamonadaceae was significantly different between gastric cancer patients and normal individuals (see Example 5).

More particularly, in one embodiment of the present invention, as a result of performing bacterial metagenomic analysis on extracellular vesicles present in subject-derived urine samples at a genus level, the content of extracellular vesicles derived from bacteria belonging to the genus Morganella, the genus Rhizobium, the genus Exiguobacterium, the genus Proteus, the genus Parabacteroides, the genus Adlercreutzia, the genus Prevotella, the genus Acinetobacter, the genus Akkermansia, the genus Oscillospira, the genus Bifidobacterium, the genus Faecalibacterium, the genus Ruminococcus, the genus Coprococcus, the genus Pediococcus, and the genus Citrobacter was significantly different between gastric cancer patients and normal individuals (see Example 5).

More particularly, in one embodiment of the present invention, as a result of performing bacterial metagenomic analysis on extracellular vesicles present in subject-derived stool samples at a phylum level, the content of extracellular vesicles derived from bacteria belonging to the phylum Tenericutes and the phylum Cyanobacteria was significantly different between gastric cancer patients and normal individuals (see Example 6).

More particularly, in one embodiment of the present invention, as a result of performing bacterial metagenomic analysis on extracellular vesicles present in subject-derived stool samples at a class level, the content of extracellular vesicles derived from bacteria belonging to the class Mollicutes was significantly different between gastric cancer patients and normal individuals (see Example 6).

More particularly, in one embodiment of the present invention, as a result of performing bacterial metagenomic analysis on extracellular vesicles present in subject-derived stool samples at an order level, the content of extracellular vesicles derived from bacteria belonging to the order RF39, the order Neisseriales, and the order Enterobacteriales was significantly different between gastric cancer patients and normal individuals (see Example 6).

More particularly, in one embodiment of the present invention, as a result of performing bacterial metagenomic analysis on extracellular vesicles present in subject-derived stool samples at a family level, the content of extracellular vesicles derived from bacteria belonging to the family Peptostreptococcaceae, the family Neisseriaceae, the family Enterobacteriaceae, the family Staphylococcaceae, the family Oxalobacteraceae, the family Moraxellaceae, and the family Planococcaceae was significantly different between gastric cancer patients and normal individuals (see Example 6).

More particularly, in one embodiment of the present invention, as a result of performing bacterial metagenomic analysis on extracellular vesicles present in subject-derived stool samples at a genus level, the content of extracellular vesicles derived from bacteria belonging to the genus Cupriavidus, the genus Proteus, the genus Methylobacterium, the genus Faecalibacterium, the genus Neisseria, the genus Staphylococcus, and the genus Acinetobacter was significantly different between gastric cancer patients and normal individuals (see Example 6).

From the above-described example results, it can be confirmed that bacteria-derived extracellular vesicles exhibiting a significant change in content in gastric cancer patients compared to normal individuals, are identified by performing bacterial metagenomic analysis on genomes present in extracellular vesicles isolated from subject-derived blood, stool, and urine, and gastric cancer may be diagnosed by analyzing an increase or decrease in the content of bacteria-derived extracellular vesicles at each level through metagenomic analysis.

Hereinafter, the present invention will be described with reference to exemplary examples to aid in understanding of the present invention. However, these examples are provided only for illustrative purposes and are not intended to limit the scope of the present invention.

### EXAMPLES

### Example 1

### Analysis of In Vivo Absorption, Distribution, and Excretion Patterns of Intestinal Bacteria and Bacteria-Derived Extracellular Vesicles

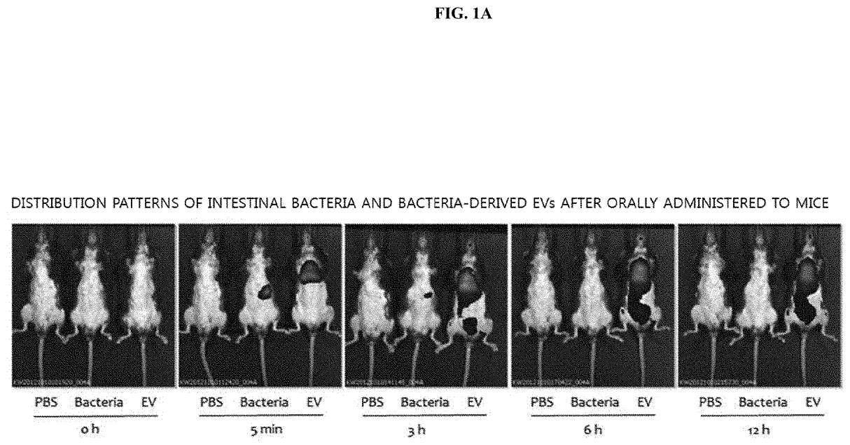

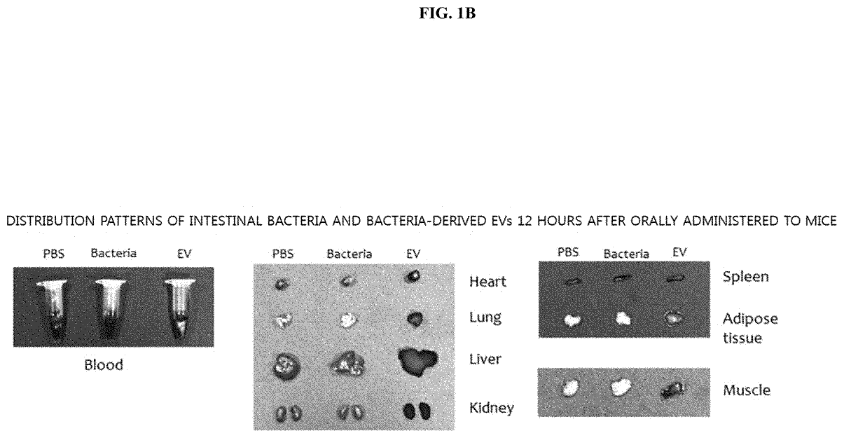

To evaluate whether intestinal bacteria and bacteria-derived extracellular vesicles are systematically absorbed through the gastrointestinal tract, an experiment was conducted using the following method. More particularly, 50 μg of each of intestinal bacteria and the bacteria-derived extracellular vesicles (EVs), labeled with fluorescence, were orally administered to the gastrointestinal tracts of mice, and fluorescence was measured at 0 h, and after 5 min, 3 h, 6 h, and 12 h. As a result of observing the entire images of mice, as illustrated in FIG. 1A, the bacteria were not systematically absorbed when administered, while the bacteria-derived EVs were systematically absorbed at 5 min after administration, and, at 3 h after administration, fluorescence was strongly observed in the bladder, from which it was confirmed that the EVs were excreted via the urinary system, and were present in the bodies up to 12 h after administration.

After intestinal bacteria and intestinal bacteria-derived extracellular vesicles were systematically absorbed, to evaluate a pattern of invasion of intestinal bacteria and the bacteria-derived EVs into various organs in the human body after being systematically absorbed, 50 μg of each of the bacteria and bacteria-derived EVs, labeled with fluorescence, were administered using the same method as that used above, and then, at 12 h after administration, blood, the heart, the lungs, the liver, the kidneys, the spleen, adipose tissue, and muscle were extracted from each mouse. As a result of observing fluorescence in the extracted tissues, as illustrated in FIG. 1B, it was confirmed that the intestinal bacteria were not absorbed into each organ, while the bacteria-derived EVs were distributed in the blood, heart, lungs, liver, kidneys, spleen, adipose tissue, and muscle.

### Example 2

### Vesicle Isolation and DNA Extraction from Blood, Stool, and Urine

To isolate extracellular vesicles and extract DNA, from blood, stool, and urine, first, blood, stool, or urine was added to a 10 ml tube and centrifuged at 3,500 ×g and 4 for 10 min to precipitate a suspension, and only a supernatant was collected, which was then placed in a new 10 ml tube. The collected supernatant was filtered using a 0.22 μm filter to remove bacteria and impurities, and then placed in centripreigugal filters (50 kD) and centrifuged at 1500 x g and 4 □ for 15 min to discard materials with a smaller size than 50 kD, and then concentrated to 10 ml. Once again, bacteria and impurities were removed therefrom using a 0.22 μm filter, and then the resulting concentrate was subjected to ultra-high speed centrifugation at 150,000×g and 4 ⊏ for 3 hours to remove a supernatant, and the agglomerated pellet was dissolved with phosphate-buffered saline (PBS), thereby obtaining vesicles.

100 μl of the extracellular vesicles isolated from the blood, stool, and urine according to the above-described method was boiled at 100 □ to allow the internal DNA to come out of the lipid and then cooled on ice. Next, the resulting vesicles were centrifuged at 10,000×g and 4 □for 30 minutes to remove the remaining suspension, only the supernatant was collected, and then the amount of DNA extracted was quantified using a NanoDrop sprectrophotometer. In addition, to verify whether bacteria-derived DNA was present in the extracted DNA, PCR was performed using 16s rDNA primers shown in Table 1 below.

TABLE 1

| | Primer | Sequence | SEQ ID NO. |
|---|---|---|---|
| 16S rDNA | 16S_V3_F | 5′-TCGTCGGCAGCGTC | 1 |
| | | AGATGTGTATAAGAG | |
| | | ACAGCCTACGGGNGG | |
| | | CWGCAG-3′ | |
| | 16S_V4_R | 5′-GTCTCGTGGGCTCG | 2 |
| | | GAGATGTGTATAAGA | |
| | | GACAGGACTACHVGG | |
| | | GTATCTAATCC-3′ | |

### Example 3

### Metagenomic Analysis Using DNA Extracted from Blood, Stool, and Urine

DNA was extracted using the same method as that used in Example 2, and then PCR was performed thereon using 16S rDNA primers shown in Table 1 to amplify DNA, followed by sequencing (Illumina MiSeq sequencer). The results were output as standard flowgram format (SFF) files, and the SFF files were converted into sequence files (.fasta) and nucleotide quality score files using GS FLX software (v2.9), and then credit rating for reads was identified, and portions with a window (20 bps) average base call accuracy of less than 99% (Phred score <20) were removed. After removing the low-quality portions, only reads having a length of 300 bps or more were used (Sickle version 1.33), and, for operational taxonomy unit (OTU) analysis, clustering was performed using UCLUST and USEARCH according to sequence similarity. In particular, clustering was performed based on sequence similarity values of 94% for genus, 90% for family, 85% for order, 80% for class, and 75% for phylum, and phylum, class, order, family, and genus levels of each OTU were classified, and bacteria with a sequence similarity of 97% or more were analyzed (QIIME) using 16S DNA sequence databases (108,453 sequences) of BLASTN and GreenGenes.

### Example 4

### Gastric Cancer Diagnostic Model Based on Metagenomic Analysis of Bacteria-Derived EVs Isolated from Blood

EVs were isolated from blood samples of 66 gastric cancer patients and 198 normal individuals, the two groups matched in age and gender, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an area under curve (AUC), sensitivity, and specificity, which are diagnostic performance indexes, were calculated by logistic regression analysis.

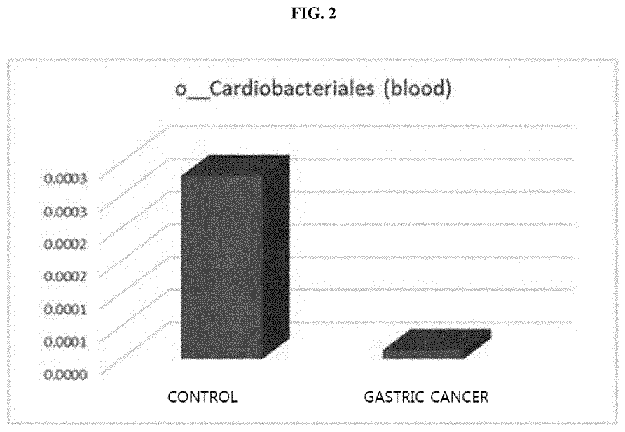

As a result of analyzing bacteria-derived EVs in blood at an order level, a diagnostic model developed using bacteria belonging to the order Cardiobacteriales as a biomarker exhibited significant diagnostic performance for gastric cancer (see Table 2 and FIG. 2).

TABLE 2

| | Control | | Gastric cancer | | | t-test | | | |
|---|---|---|---|---|---|---|---|---|---|
| | Mean | SD | Mean | SD | Ratio | p-value | AUC | sensitivity | specificity |
| o Cardiobacteriales | 0.0003 | 0.0009 | 0.0000 | 0.0001 | 0.05 | 0.00012 | 0.57 | 0.15 | 0.95 |

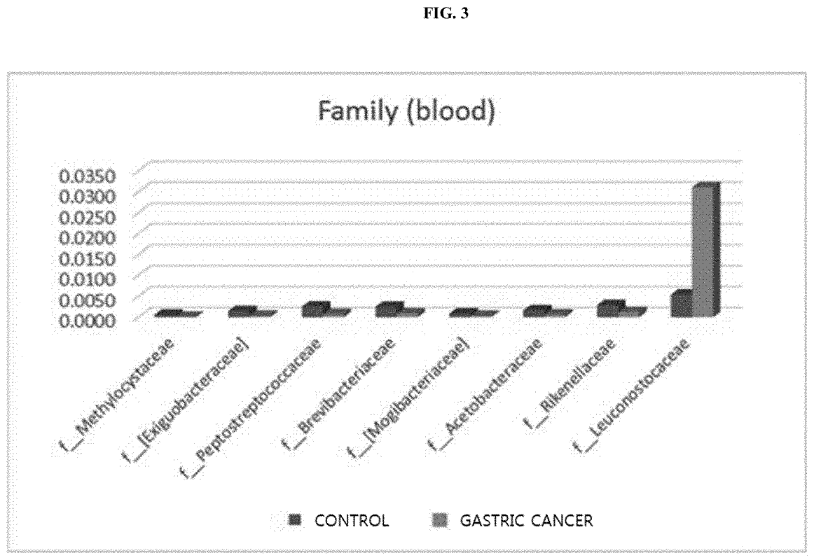

As a result of analyzing bacteria-derived EVs in blood at a family level, a diagnostic model developed using, as a biomarker, one or more bacteria selected from the family Methylocystaceae, the family Exiguobacteraceae, the family Peptostreptococcaceae, the family Brevibacteriaceae, the family Mogibacteriaceae, the family Acetobacteraceae, the family Rikenellaceae, and the family Leuconostocaceae exhibited significant diagnostic performance for gastric cancer (see Table 3 and FIG. 3).

TABLE 3

| | Control | | Gastric cancer | | | t-test | | | |
|---|---|---|---|---|---|---|---|---|---|
| | Mean | SD | Mean | SD | Ratio | p-value | AUC | sensitivity | specificity |
| f_Methylocystaceae | 0.0005 | 0.0019 | 0.0000 | 0.0001 | 0.08 | 0.00067 | 0.58 | 0.17 | 0.88 |
| f_[Exiguobacteraceae] | 0.0014 | 0.0057 | 0.0003 | 0.0009 | 0.21 | 0.00847 | 0.50 | 0.07 | 0.98 |
| f_Peptostreptococcaceae | 0.0025 | 0.0069 | 0.0007 | 0.0015 | 0.29 | 0.00102 | 0.65 | 0.32 | 0.88 |
| f_Brevibacteriaceae | 0.0024 | 0.0072 | 0.0008 | 0.0019 | 0.35 | 0.00563 | 0.58 | 0.17 | 0.92 |
| f_[Mogibacteriaceae] | 0.0008 | 0.0022 | 0.0003 | 0.0005 | 0.35 | 0.00142 | 0.54 | 0.19 | 0.91 |
| f_Acetobacteraceae | 0.0016 | 0.0035 | 0.0006 | 0.0010 | 0.36 | 0.00042 | 0.57 | 0.15 | 0.95 |
| f_Rikenellaceae | 0.0028 | 0.0063 | 0.0012 | 0.0023 | 0.42 | 0.00214 | 0.56 | 0.20 | 0.94 |
| f_Leuconostocaceae | 0.0054 | 0.0083 | 0.0311 | 0.0473 | 5.78 | 0.00004 | 0.62 | 0.98 | 0.32 |

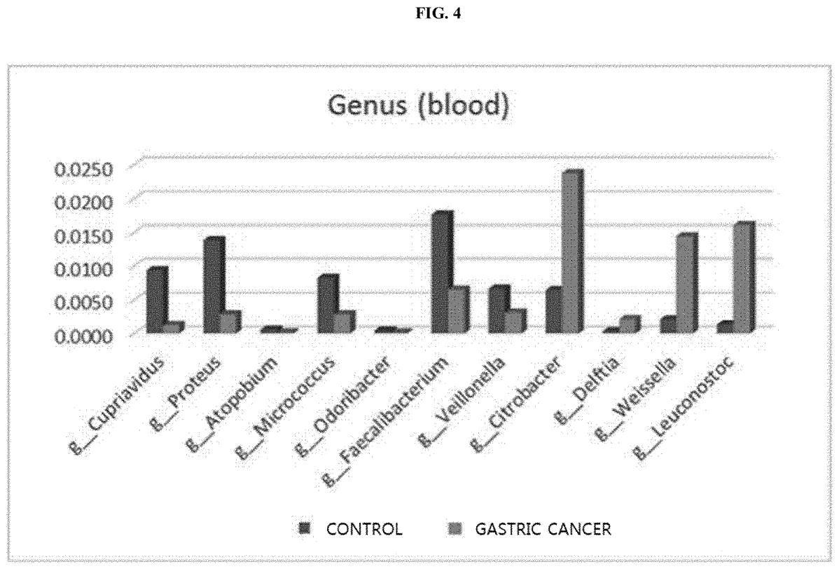

As a result of analyzing bacteria-derived EVs in blood at a genus level, a diagnostic model developed using, as a biomarker, one or more bacteria selected from the genus Cupriavidus, the genus Proteus, the genus Atopobium, the genus Micrococcus, the genus Odoribacter, the genus Faecalibacterium, the genus Veillonella, the genus Citrobacter, the genus Delftia, the genus Delftia, and the genus Leuconostoc exhibited significant diagnostic performance for gastric cancer (see Table 4 and FIG. 4).

TABLE 4

| | Control | | Gastric cancer | | | t-test | | | |
|---|---|---|---|---|---|---|---|---|---|
| | Mean | SD | Mean | SD | Ratio | p-value | AUC | sensitivity | specificity |
| g_Cupriavidus | 0.0094 | 0.0158 | 0.0013 | 0.0026 | 0.13 | 0.00000 | 0.85 | 0.78 | 0.85 |
| g_Proteus | 0.0138 | 0.0298 | 0.0028 | 0.0051 | 0.21 | 0.00000 | 0.61 | 0.35 | 0.85 |
| g_Atopobium | 0.0006 | 0.0013 | 0.0002 | 0.0004 | 0.29 | 0.00010 | 0.65 | 0.67 | 0.44 |
| g_Micrococcus | 0.0082 | 0.0115 | 0.0029 | 0.0051 | 0.35 | 0.00000 | 0.65 | 0.52 | 0.67 |
| g_Odoribacter | 0.0004 | 0.0014 | 0.0002 | 0.0003 | 0.36 | 0.00927 | 0.63 | 0.48 | 0.77 |
| g_Faecalibacterium | 0.0176 | 0.0243 | 0.0065 | 0.0090 | 0.37 | 0.00000 | 0.64 | 0.39 | 0.90 |
| g_Veillonella | 0.0066 | 0.0122 | 0.0031 | 0.0043 | 0.47 | 0.00061 | 0.69 | 0.54 | 0.64 |
| g_Citrobacter | 0.0065 | 0.0096 | 0.0238 | 0.0373 | 3.68 | 0.00040 | 0.73 | 0.98 | 0.33 |
| g_Delftia | 0.0004 | 0.0011 | 0.0021 | 0.0034 | 5.95 | 0.00010 | 0.64 | 0.38 | 0.87 |
| g_Weissella | 0.0021 | 0.0052 | 0.0144 | 0.0230 | 6.90 | 0.00005 | 0.69 | 0.55 | 0.67 |
| g_Leuconostoc | 0.0014 | 0.0051 | 0.0161 | 0.0271 | 11.79 | 0.00006 | 0.61 | 0.58 | 0.56 |

### Example 5

### Gastric Cancer Diagnostic Model Based on Metagenomic

Analysis of Bacteria-Derived EVs Isolated from Urine

EVs were isolated from urine samples of 61 gastric cancer patients and 120 normal individuals, the two groups matched in age and gender, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an AUC, sensitivity, and specificity, which are diagnostic performance indexes, were calculated by logistic regression analysis.

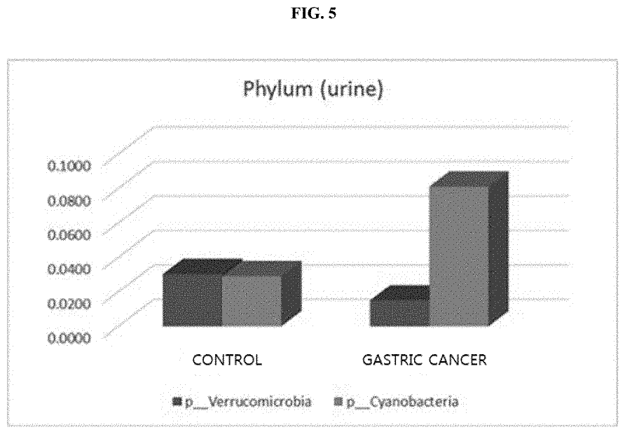

As a result of analyzing bacteria-derived EVs in urine at a phylum level, a diagnostic model developed using bacteria belonging to the phylum Verrucomicrobia and the phylum Cyanobacteria as a biomarker exhibited significant diagnostic performance for gastric cancer (see Table 5 and FIG. 5).

TABLE 5

| | Control | | Gastric cancer | | | t-test | | | |
|---|---|---|---|---|---|---|---|---|---|
| | Mean | SD | Mean | SD | Ratio | p-value | AUC | sensitivity | specificity |
| p_Verrucomicrobia | 0.0303 | 0.0358 | 0.0149 | 0.0173 | 0.49 | 0.00042 | 0.64 | 0.80 | 0.33 |
| p_Cyanobacteria | 0.0291 | 0.0397 | 0.0810 | 0.1400 | 2.79 | 0.00512 | 0.52 | 0.97 | 0.19 |

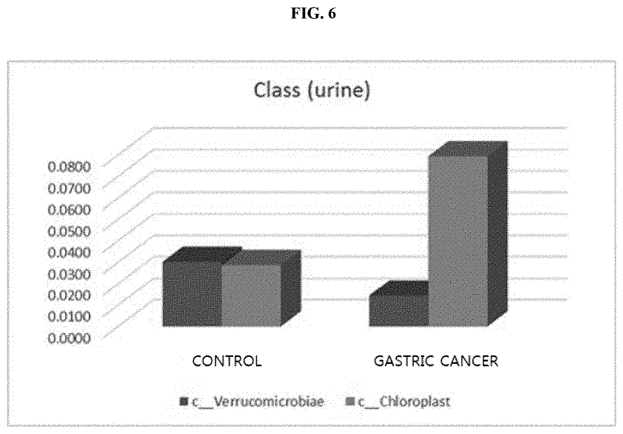

As a result of analyzing bacteria-derived EVs in urine at a class level, a diagnostic model developed using, as a biomarker, one or more bacteria selected from the class Verrucomicrobiae and the class Chloroplast exhibited significant diagnostic performance for gastric cancer (see Table 6 and FIG. 6).

TABLE 6

| | Control | | Gastric cancer | | | t-test | | | |
|---|---|---|---|---|---|---|---|---|---|
| | Mean | SD | Mean | SD | Ratio | p-value | AUC | sensitivity | specificity |
| c_Verrucomicrobiae | 0.0301 | 0.0356 | 0.0144 | 0.0174 | 0.48 | 0.00031 | 0.65 | 0.79 | 0.38 |
| p_Chloroplast | 0.0286 | 0.0396 | 0.0793 | 0.1386 | 2.77 | 0.00581 | 0.52 | 0.97 | 0.19 |

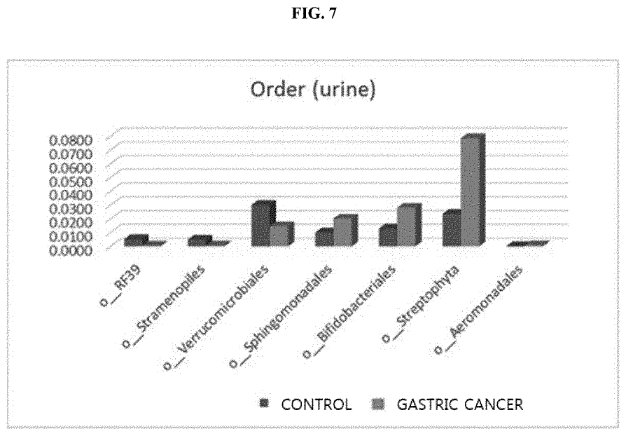

As a result of analyzing bacteria-dervied EVs in urine at an order level, a diagnostic model developed using, as a biomarker, one or more bacteria selected from the order RF39, the order Stramenopiles, the order Verrucomicrobiales, the order Sphingomonadales, the order Bifidobacteriales, the order Streptophyta, and the order Aeromonadales exhibited significant diagnostic performance for gastric cancer (see Table 7 and FIG. 7).

TABLE 7

| | Control | | Gastric cancer | | | t-test | | | |
|---|---|---|---|---|---|---|---|---|---|
| | Mean | SD | Mean | SD | Ratio | p-value | AUC | sensitivity | specificity |
| o_RF39 | 0.0052 | 0.0115 | 0.0006 | 0.0009 | 0.11 | 0.00017 | 0.73 | 0.54 | 0.78 |
| o_Stramenopiles | 0.0049 | 0.0080 | 0.0006 | 0.0016 | 0.13 | 0.00000 | 0.57 | 0.96 | 0.13 |
| o_Verrucomicrobiales | 0.0301 | 0.0356 | 0.0144 | 0.0174 | 0.48 | 0.00031 | 0.65 | 0.79 | 0.38 |
| o_Sphingomonadales | 0.0100 | 0.0089 | 0.0202 | 0.0236 | 2.02 | 0.00147 | 0.60 | 0.93 | 0.33 |
| o_Bifidobacteriales | 0.0129 | 0.0175 | 0.0281 | 0.0355 | 2.17 | 0.00217 | 0.60 | 0.93 | 0.27 |
| o_Streptophyta | 0.0237 | 0.0378 | 0.0785 | 0.1383 | 3.30 | 0.00289 | 0.55 | 0.96 | 0.22 |
| o_Aeromonadales | 0.0002 | 0.0005 | 0.0007 | 0.0018 | 4.19 | 0.01455 | 0.61 | 0.95 | 0.17 |

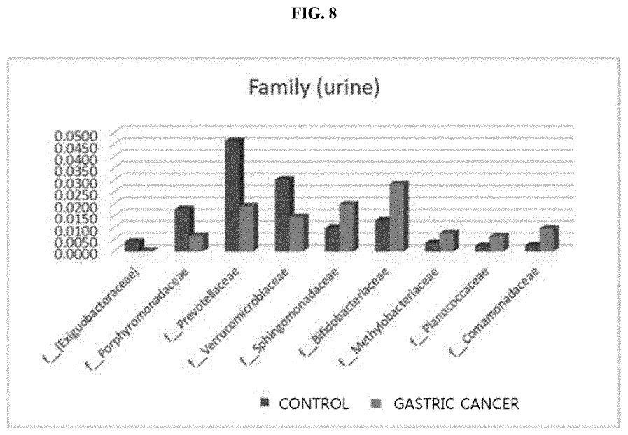

As a result of analyzing bacteria-derived EVs in urine at a family level, a diagnostic model developed using, as a biomarker, one or more bacteria selected from the family Exiguobacteraceae, the family Porphyromonadaceae, the family Prevotellaceae, the family Verrucomicrobiaceae, the family Sphingomonadaceae, the family Bifidobacteriaceae, the family Methylobacteriaceae, the family Planococcaceae, and the family Comamonadaceae exhibited significant diagnostic performance for gastric cancer (see Table 8 and FIG. 8).

TABLE 8

| | Control | | Gastric cancer | | | t-test | | | |
|---|---|---|---|---|---|---|---|---|---|
| | Mean | SD | Mean | SD | Ratio | p-value | AUC | sensitivity | specificity |
| f_[Exiguobacteraceae] | 0.0039 | 0.0106 | 0.0002 | 0.0006 | 0.05 | 0.00098 | 0.56 | 0.88 | 0.22 |
| f_Porphyromonadaceae | 0.0177 | 0.0187 | 0.0063 | 0.0109 | 0.36 | 0.00000 | 0.61 | 0.94 | 0.22 |
| f_Prevotellaceae | 0.0464 | 0.0728 | 0.0188 | 0.0140 | 0.41 | 0.00051 | 0.48 | 0.33 | 0.80 |
| f_Verrucomicrobiaceae | 0.0301 | 0.0356 | 0.0144 | 0.0174 | 0.48 | 0.00031 | 0.65 | 0.79 | 0.38 |
| f_Sphingomonadaceae | 0.0098 | 0.0088 | 0.0196 | 0.0231 | 2.01 | 0.00170 | 0.60 | 0.93 | 0.34 |
| f_Bifidobacteriaceae | 0.0129 | 0.0175 | 0.0281 | 0.0355 | 2.17 | 0.00217 | 0.34 | 0.15 | 0.81 |
| f_Methylobacteriaceae | 0.0034 | 0.0046 | 0.0075 | 0.0102 | 2.20 | 0.00353 | 0.54 | 0.92 | 0.22 |
| f_Planococcaceae | 0.0022 | 0.0034 | 0.0062 | 0.0083 | 2.83 | 0.00043 | 0.50 | 0.99 | 0.03 |
| f_Comamonadaceae | 0.0024 | 0.0032 | 0.0095 | 0.0164 | 4.02 | 0.00098 | 0.53 | 0.26 | 0.72 |

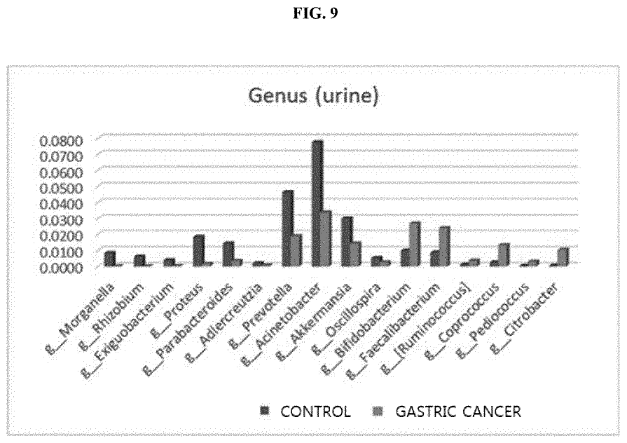

As a result of analyzing bacteria-derived EVs in urine at a genus level, a diagnostic model developed using, as a biomarker, one or more bacteria selected from the genus Morganella, the genus Rhizobium, the genus Exiguobacterium, the genus Proteus, the genus Parabacteroides, the genus Adlercreutzia, the genus Prevotella, the genus Acinetobacter, the genus Akkermansia, the genus Oscillospira, the genus Bifidobacterium, the genus Faecalibacterium, the genus Ruminococcus, the genus Coprococcus, the genus Pediococcus, and the genus Citrobacter exhibited significant diagnostic performance for gastric cancer (see Table 9 and FIG. 9).

TABLE 9

| | Control | | Gastric cancer | | | t-test | | | |
|---|---|---|---|---|---|---|---|---|---|
| | Mean | SD | Mean | SD | Ratio | p-value | AUC | sensitivity | specificity |
| g_Morganella | 0.0082 | 0.0217 | 0.0000 | 0.0001 | 0.00 | 0.00038 | 0.53 | 0.16 | 0.88 |
| g_Rhizobium | 0.0060 | 0.0063 | 0.0001 | 0.0002 | 0.01 | 0.00000 | 0.66 | 0.92 | 0.33 |
| g_Exiguobacterium | 0.0039 | 0.0106 | 0.0002 | 0.0006 | 0.05 | 0.00100 | 0.60 | 0.95 | 0.11 |
| g_Proteus | 0.0184 | 0.0212 | 0.0015 | 0.0031 | 0.08 | 0.00000 | 0.49 | 0.99 | 0.14 |
| g_Parabacteroides | 0.0143 | 0.0179 | 0.0034 | 0.0056 | 0.24 | 0.00000 | 0.61 | 0.95 | 0.19 |
| g_Acllercreutzia | 0.0020 | 0.0042 | 0.0007 | 0.0009 | 0.33 | 0.00350 | 0.57 | 0.94 | 0.20 |
| g_Prevotella | 0.0464 | 0.0728 | 0.0188 | 0.0140 | 0.41 | 0.00051 | 0.48 | 0.33 | 0.80 |
| g_Acinetobacter | 0.0776 | 0.1128 | 0.0338 | 0.0407 | 0.44 | 0.00073 | 0.56 | 0.29 | 0.80 |
| g_Akkermansia | 0.0299 | 0.0355 | 0.0143 | 0.0171 | 0.48 | 0.00031 | 0.65 | 0.78 | 0.38 |
| g_Oscillospira | 0.0052 | 0.0053 | 0.0025 | 0.0039 | 0.49 | 0.00034 | 0.72 | 0.72 | 0.61 |
| g_Bifidobacterium | 0.0099 | 0.0120 | 0.0267 | 0.0355 | 2.70 | 0.00050 | 0.61 | 0.95 | 0.27 |
| g_Faecalibacterium | 0.0087 | 0.0140 | 0.0239 | 0.0397 | 2.74 | 0.00438 | 0.60 | 0.93 | 0.28 |
| g_[Ruminococcus] | 0.0012 | 0.0017 | 0.0036 | 0.0050 | 3.10 | 0.00034 | 0.71 | 0.45 | 0.84 |
| g_Coprococcus | 0.0025 | 0.0035 | 0.0132 | 0.0212 | 5.31 | 0.00017 | 0.57 | 0.32 | 0.81 |
| g_Pediococcus | 0.0003 | 0.0013 | 0.0030 | 0.0042 | 8.54 | 0.00001 | 0.50 | 0.55 | 0.48 |
| g_Citrobacter | 0.0005 | 0.0013 | 0.0103 | 0.0235 | 20.12 | 0.00147 | 0.56 | 0.96 | 0.08 |

### Example 6

### Gastric Cancer Diagnostic Model Based on Metagenomic Analysis of Bacteria-Derived EVs Isolated from Stool

EVs were isolated from stool samples of 63 gastric cancer patients and 126 normal individuals, the two groups matched in age and gender, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an AUC, sensitivity, and specificity, which are diagnostic performance indexes, were calculated by logistic regression analysis.

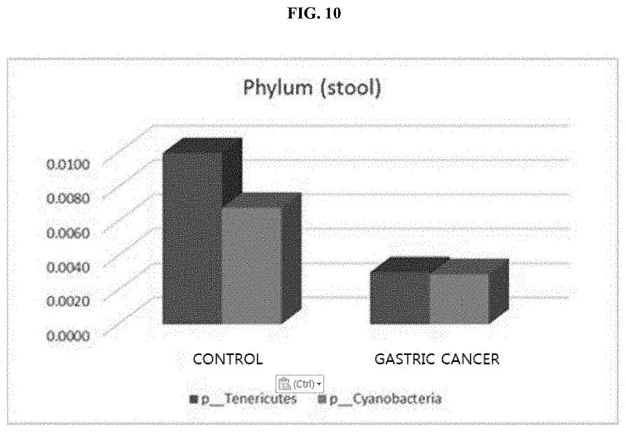

As a result of analyzing bacteria-derived EVs in stool at a phylum level, a diagnostic model developed using bacteria belonging to the phylum Tenericutes and the phylum Cyanobacteria as a biomarker exhibited significant diagnostic performance for gastric cancer (see Table 10 and FIG. 10).

TABLE 10

| | Control | | Gastric cancer | | t-test | | | | |
|---|---|---|---|---|---|---|---|---|---|
| | Mean | SD | Mean | SD | p-value | Ratio | AUC | sensitivity | specificity |
| p_Tenericutes | 0.0100 | 0.0257 | 0.0030 | 0.0072 | 0.0000 | 0.30 | 0.78 | 1.00 | 0.11 |
| p_Cyanobacteria | 0.0068 | 0.0223 | 0.0029 | 0.0054 | 0.0054 | 0.43 | 0.78 | 1.00 | 0.08 |

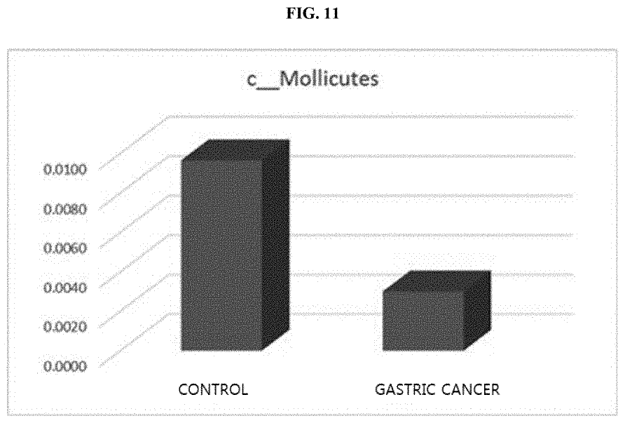

As a result of analyzing bacteria-derived EVs in stool at a class level, a diagnostic model developed using, as a biomarker, one or more bacteria selected from the class Mollicutes exhibited significant diagnostic performance for gastric cancer (see Table 11 and FIG. 11).

TABLE 11

| | Control | | Gastric cancer | | t-test | | | | |
|---|---|---|---|---|---|---|---|---|---|
| Taxon | Mean | SD | Mean | SD | p-value | Ratio | AUC | sensitivity | specificity |
| c_Mollicutes | 0.0096 | 0.0256 | 0.0030 | 0.0071 | 0.0001 | 0.31 | 0.78 | 1.00 | 0.11 |

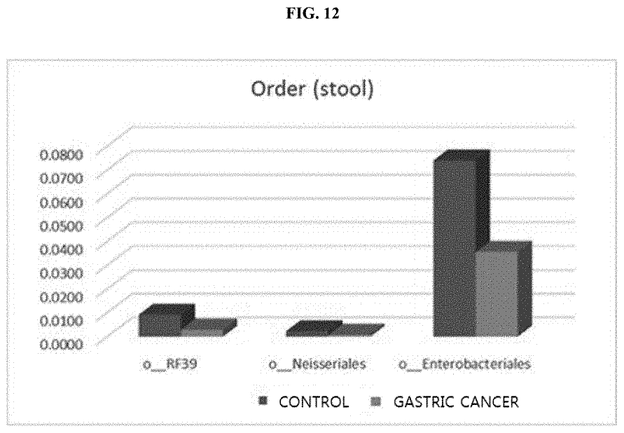

As a result of analyzing bacteria-derived EVs in stool at an order level, a diagnostic model developed using, as a biomarker, one or more bacteria selected from the order RF39, the order Neisseriales, and the order Enterobacteriales exhibited significant diagnostic performance for gastric cancer (see Table 12 and FIG. 12).

TABLE 12

| | Control | | Gastric cancer | | t-test | | | | |
|---|---|---|---|---|---|---|---|---|---|
| Taxon | Mean | SD | Mean | SD | p-value | Ratio | AUC | sensitivity | specificity |
| o_RF39 | 0.0091 | 0.0250 | 0.0029 | 0.0071 | 0.0001 | 0.32 | 0.78 | 1.00 | 0.11 |
| o_Neisseriales | 0.0022 | 0.0045 | 0.0008 | 0.0014 | 0.000 | 0.35 | 0.80 | 0.98 | 0.17 |
| o_Enterobacteriales | 0.0740 | 0.1133 | 0.0356 | 0.0481 | 0.0000 | 0.48 | 0.79 | 0.99 | 0.10 |

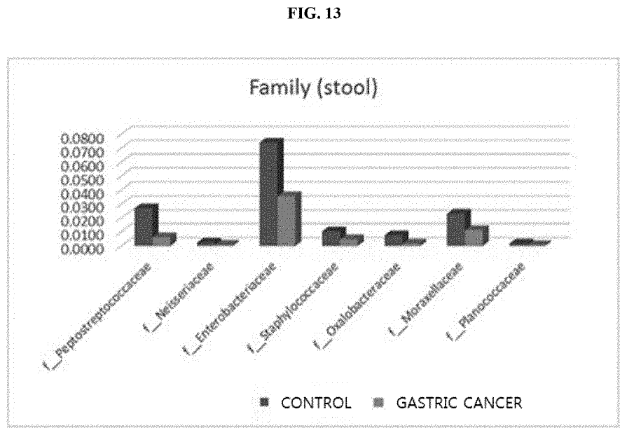

As a result of analyzing bacteria-derived EVs in stool at a family level, a diagnostic model developed using, as a biomarker, one or more bacteria selected from the family Peptostreptococcaceae, the family Neisseriaceae, the family Enterobacteriaceae, the family Staphylococcaceae, the family Oxalobacteraceae, the family Moraxellaceae, and the family Planococcaceae exhibited significant diagnostic performance for gastric cancer (see Table 13 and FIG. 13).

TABLE 13

| | Control | | Gastric cancer | | t-test | | | | |
|---|---|---|---|---|---|---|---|---|---|
| Taxon | Mean | SD | Mean | SD | p-value | Ratio | AUC | sensitivity | specificity |
| f_Peptostreptococcaceae | 0.0270 | 0.0617 | 0.0062 | 0.0275 | 0.0000 | 0.23 | 0.84 | 0.99 | 0.08 |
| f_Neisseriaceae | 0.0022 | 0.0045 | 0.0008 | 0.0014 | 0.0000 | 0.35 | 0.80 | 0.98 | 0.17 |
| f_Enternbacteriaceae | 0.0740 | 0.1133 | 0.0356 | 0.0481 | 0.0000 | 0.48 | 0.79 | 0.99 | 0.10 |
| f_Staphylococcaceae | 0.0103 | 0.0189 | 0.0047 | 0.0073 | 0.0000 | 0.45 | 0.79 | 0.99 | 0.10 |
| f_Oxalobacteraceae | 0.0075 | 0.0339 | 0.0016 | 0.0024 | 0.0014 | 0.21 | 0.79 | 0.99 | 0.08 |
| f_Moraxellaceae | 0.0232 | 0.0440 | 0.0112 | 0.0135 | 0.0000 | 0.48 | 0.78 | 0.99 | 0.10 |
| f_Planococcaceae | 0.0016 | 0.0063 | 0.0005 | 0.0012 | 0.0032 | 0.33 | 0.78 | 0.99 | 0.08 |

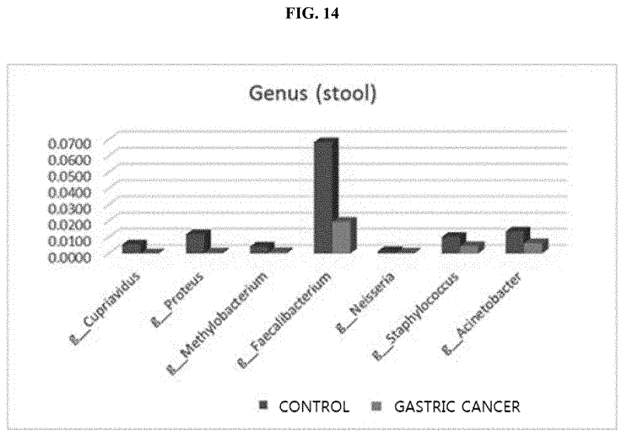

As a result of analyzing bacteria-derived EVs in stool at a genus level, a diagnostic model developed using, as a biomarker, one or more bacteria selected from the genus Cupriavidus, the genus Proteus, the genus Methylobacterium, the genus Faecalibacterium, the genus Neisseria, the genus Staphylococcus, and the genus Acinetobacter exhibited significant diagnostic performance for gastric cancer (see Table 14 and FIG. 14).

TABLE 14

| | Control | | Gastric cancer | | t-test | | | | |
|---|---|---|---|---|---|---|---|---|---|
| Taxon | Mean | SD | Mean | SD | p-value | Ratio | AUC | sensitivity | specificity |
| g_Cupriavidus | 0.0054 | 0.0308 | 0.0000 | 0.0001 | 0.0011 | 0.01 | 0.83 | 0.97 | 0.21 |
| g_Proteus | 0.0117 | 0.0265 | 0.0005 | 0.0018 | 0.0000 | 0.04 | 0.83 | 0.98 | 0.16 |
| g_Methylobacterium | 0.0041 | 0.0184 | 0.0007 | 0.0016 | 0.0007 | 0.16 | 0.78 | 1.00 | 0.10 |
| g_Faecalibacterium | 0.0684 | 0.0897 | 0.0194 | 0.0282 | 0.0000 | 0.28 | 0.84 | 0.97 | 0.24 |
| g_Neisseria | 0.0013 | 0.0037 | 0.0004 | 0.0010 | 0.0002 | 0.33 | 0.79 | 0.99 | 0.08 |
| g_Staphylococcus | 0.0100 | 0.0188 | 0.0044 | 0.0068 | 0.0000 | 0.44 | 0.79 | 0.99 | 0.10 |
| g_Acinetobacter | 0.0134 | 0.0222 | 0.0063 | 0.0073 | 0.0000 | 0.47 | 0.79 | 0.99 | 0.11 |

The above description of the present invention is provided only for illustrative purposes, and it will be understood by one of ordinary skill in the art to which the present invention pertains that the invention may be embodied in various modified forms without departing from the spirit or essential characteristics thereof. Thus, the embodiments described herein should be considered in an illustrative sense only and not for the purpose of limitation.

### INDUSTRIAL APPLICABILITY

A method of providing information for gastric cancer diagnosis through bacterial metagenomic analysis, according to the present invention, can be used to predict a risk for gastric cancer and diagnose gastric cancer by analyzing an increase or decrease in content of extracellular vesicles derived from specific bacteria through bacterial metagenomic analysis of a genome present in extracellular vesicles isolated from a subject-derived sample. Extracellular vesicles secreted from bacteria existing in the environment are absorbed into the human body, and thus may directly affect the occurrence of cancer, and gastric cancer has a very high incidence rate and a very high mortality rate in Korea, and thus prevention and early diagnosis thereof through prediction of the onset thereof is very important. Thus, according to the present disclosure, a risk for gastric cancer can be predicted through bacterial metagenomic analysis of a genome present in a human body-derived sample, and thus the onset of gastric cancer can be delayed or gastric cancer can be prevented through appropriate management by early diagnosis and prediction of a risk group for gastric cancer, and, even after gastric cancer occurs, early diagnosis for gastric cancer can be implemented, thereby lowering a disease rate and increasing therapeutic effects. In addition, patients diagnosed with gastric cancer are able to avoid exposure to causative factors predicted by bacterial metagenomic analysis according to the present invention, whereby the progression of gastric cancer can be ameliorated, or recurrence of gastric cancer can be prevented.

1. A method of providing information for gastric cancer diagnosis, the method comprising:
(a) extracting DNA from extracellular vesicles isolated from a subject sample;
(b) performing polymerase chain reaction (PCR) on the extracted DNA using a pair of primers having SEQ ID NO:1 and SEQ ID NO: 2; and
(c) comparing an increase or decrease in content of bacteria-derived extracellular vesicles of the subject sample with that of a normal individual-derived sample through sequencing of a product of the PCR.

2. The method of claim 1, wherein the comparing comprises comparing an increase or decrease in content of extracellular vesicles derived from one or more bacteria selected from the group consisting of the phylum Verrucomicrobia and the phylum Cyanobacteria that are isolated from a subject urine sample; and the phylum Tenericutes and the phylum Cyanobacteria that are isolated from a subject stool sample.

3. The method of claim 1, wherein the comparing comprises comparing an increase or decrease in content of extracellular vesicles derived from one or more bacteria selected from the group consisting of the class Verrucomicrobiae and the class Chloroplast that are isolated from a subject urine sample; and the class Mollicutes that is isolated from a subject stool sample.

4. The method of claim 1, wherein the comparing comprises comparing an increase or decrease in content of extracellular vesicles derived from one or more bacteria selected from the group consisting of the order Cardiobacteriales that is isolated from a subject blood sample; the order RF39, the order Stramenopiles, the order Verrucomicrobiales, the order Sphingomonadales, the order Bifidobacteriales, the order Streptophyta, and the order Aeromonadales that are isolated from a subject urine sample; and the order RF39, the order Neisseriales, and the order Enterobacteriales that are isolated from a subject stool sample.

5. The method of claim 1, wherein the comparing comprises comparing an increase or decrease in content of extracellular vesicles derived from one or more bacteria selected from the group consisting of the family Methylocystaceae, the family Exiguobacteraceae, the family Peptostreptococcaceae, the family Brevibacteriaceae, the family Mogibacteriaceae, the family Acetobacteraceae, the family Rikenellaceae, and the family Leuconostocaceae that are isolated from a subject blood sample; the family Exiguobacteraceae, the family Porphyromonadaceae, the family Prevotellaceae, the family Verrucomicrobiaceae, the family Sphingomonadaceae, the family Bifidobacteriaceae, the family Methylobacteriaceae, the family Planococcaceae, and the family Comamonadaceae that are isolated from a subject urine sample; and the family Peptostreptococcaceae, the family Neisseriaceae, the family Enterobacteriaceae, the family Staphylococcaceae, the family Oxalobacteraceae, the family Moraxellaceae, and the family Planococcaceae that are isolated from a subject stool sample.

6. The method of claim 1, wherein the comparing comprises comparing an increase or decrease in content of extracellular vesicles derived from one or more bacteria selected from the group consisting of the genus Cupriavidus, the genus Proteus, the genus Atopobium, the genus Micrococcus, the genus Odoribacter, the genus Faecalibacterium, the genus Veillonella, the genus Citrobacter, the genus Delftia, the genus Delftia, and the genus Leuconostoc that are isolated from a subject blood sample; the genus Morganella, the genus Rhizobium, the genus Exiguobacterium, the genus Proteus, the genus Parabacteroides, the genus Adlercreutzia, the genus Prevotella, the genus Acinetobacter, the genus Akkermansia, the genus Oscillospira, the genus Bifidobacterium, the genus Faecalibacterium, the genus Ruminococcus, the genus Coprococcus, the genus Pediococcus, and the genus Citrobacter that are isolated from a subject urine sample; and the genus Cupriavidus, the genus Proteus, the genus Methylobacterium, the genus Faecalibacterium, the genus Neisseria, the genus Staphylococcus, and the genus Acinetobacter that are isolated from a subject stool sample.

7. The method of claim 1, wherein the subject sample is blood, urine, or stool.

8. The method of claim 7, wherein the blood is whole blood, serum, plasma, or blood mononuclear cells.

9. A method of diagnosing gastric cancer, the method comprising the following processes:
(a) extracting DNA from extracellular vesicles isolated from a subject sample;
(b) performing polymerase chain reaction (PCR) on the extracted DNA using a pair of primers having SEQ ID NO:1 and SEQ ID NO: 2; and
(c) comparing an increase or decrease in content of bacteria-derived extracellular vesicles of the subject sample with that of a normal individual-derived sample through sequencing of a product of the PCR.

10. The method of claim 9, wherein the comparing comprises comparing an increase or decrease in content of extracellular vesicles derived from one or more bacteria selected from the group consisting of the phylum Verrucomicrobia and the phylum Cyanobacteria that are isolated from a subject urine sample; and the phylum Tenericutes and the phylum Cyanobacteria that are isolated from a subject stool sample.

11. The method of claim 9, wherein the comparing comprises comparing an increase or decrease in content of extracellular vesicles derived from one or more bacteria selected from the group consisting of the class Verrucomicrobiae and the class Chloroplast that are isolated from a subject urine sample; and the class Mollicutes that is isolated from a subject stool sample.

12. The method of claim 9, wherein the comparing comprises comparing an increase or decrease in content of extracellular vesicles derived from one or more bacteria selected from the group consisting of the order Cardiobacteriales that is isolated from a subject blood sample; the order RF39, the order Stramenopiles, the order Verrucomicrobiales, the order Sphingomonadales, the order Bifidobacteriales, the order Streptophyta, and the order Aeromonadales that are isolated from a subject urine sample; and the order RF39, the order Neisseriales, and the order Enterobacteriales that are isolated from a subject stool sample.

13. The method of claim 9, wherein the comparing comprises comparing an increase or decrease in content of extracellular vesicles derived from one or more bacteria selected from the group consisting of the family Methylocystaceae, the family Exiguobacteraceae, the family Peptostreptococcaceae, the family Brevibacteriaceae, the family Mogibacteriaceae, the family Acetobacteraceae, the family Rikenellaceae, and the family Leuconostocaceae that are isolated from a subject blood sample; the family Exiguobacteraceae, the family Porphyromonadaceae, the family Prevotellaceae, the family Verrucomicrobiaceae, the family Sphingomonadaceae, the family Bifidobacteriaceae, the family Methylobacteriaceae, the family Planococcaceae, and the family Comamonadaceae that are isolated from a subject urine sample; and the family Peptostreptococcaceae, the family Neisseriaceae, the family Enterobacteriaceae, the family Staphylococcaceae, the family Oxalobacteraceae, the family Moraxellaceae, and the family Planococcaceae that are isolated from a subject stool sample.

14. The method of claim 9, wherein the comparing comprises comparing an increase or decrease in content of extracellular vesicles derived from one or more bacteria selected from the group consisting of the genus Cupriavidus, the genus Proteus, the genus Atopobium, the genus Micrococcus, the genus Odoribacter, the genus Faecalibacterium, the genus Veillonella, the genus Citrobacter, the genus Delftia, the genus Delftia, and the genus Leuconostoc that are isolated from a subject blood sample; the genus Morganella, the genus Rhizobium, the genus Exiguobacterium, the genus Proteus, the genus Parabacteroides, the genus Adlercreutzia, the genus Prevotella, the genus Acinetobacter, the genus Akkermansia, the genus Oscillospira, the genus Bifidobacterium, the genus Faecalibacterium, the genus Ruminococcus, the genus Coprococcus, the genus Pediococcus, and the genus Citrobacter that are isolated from a subject urine sample; and the genus Cupriavidus, the genus Proteus, the genus Methylobacterium, the genus Faecalibacterium, the genus Neisseria, the genus Staphylococcus, and the genus Acinetobacter that are isolated from a subject stool sample.

15. The method of claim 9, wherein the subject sample is blood, urine, or stool.

16. The method of claim 15, wherein the blood is whole blood, serum, plasma, or blood mononuclear cells.
